(19) United States
(12) Patent Application Publication
(10) Pub. No.: US 2007/0244468 A1
(43) Pub. Date: Oct. 18, 2007

(54) Safety Device for Use with Catheters

(76) Inventor: Nick Kostandaras, Richfield, OH (US)

Correspondence Address: RANKIN, HILL, PORTER & CLARK, LLP, 925 EUCLID AVENUE, SUITE 700, CLEVELAND, OH, 44115-1405, US

(21) Appl. No.: 11/279,635

(22) Filed: Apr. 13, 2006

(51) Int. Cl. A61M 25/00

(52) U.S. Cl. 604/523

(57) ABSTRACT

A safety device for use with a catheter to which a discharge conduit is connected includes a flexible, pleated segment that permits the discharge conduit to be temporarily axially elongated if sudden or excessive force is applied thereto. The pleated segment also permits limited torsional movement of the conduit. The safety device preferably includes a rotatable, fluid-tight connector that permits relative rotation between those elements to which it is connected.

### BACKGROUND OF THE INVENTION

1. Field of the Invention.

The invention relates to catheters and, more particularly, to a safety device that can be used with a catheter, such as a urinary catheter, that will result in a reduced tendency to cause pain to the user upon the application of sudden or forceful movements.

2. Description of the Prior Art.

A typical internal, or indwelling, urinary catheter is known as a Foley catheter. Such catheters consist of a hollow tube having a tip at one end which is inserted into the body. The tip has one or more openings that communicate with the interior of the hollow tube. The other end of the tube is disposed externally of the body and is connected to a waste receptacle by means of a flexible discharge conduit. An annular inflatable portion, or balloon, is spaced inwardly from the tip. The balloon is deflated during placement of the catheter into the body and thereafter is inflated after the tip is properly positioned within the body in the urinary bladder.

In order to inflate the balloon, the external portion of the catheter is provided with a branch arm having a one-way valve structure through which fluid such as water or air under pressure may be admitted. The valve communicates with the inflatable balloon through a narrow passageway lying substantially parallel to the hollow tube. By insertion of either water or air under pressure in this manner, the balloon, when inflated, assumes a generally toroidal shape. In the example where the catheter is inserted into the urinary bladder, the inflated toroid serves to retain the tip of the catheter at the entrance of the urinary bladder. Such placement of the tip assures proper drainage of the bladder through the openings provided at the tip of the hollow tube.

The waste receptacle typically is a small, flexible bag made of a plastics material that is secured to the patient's leg by straps or tape to permit ambulatory movement of the patient. Alternatively, the waste receptacle may be mounted to a structure that supports the patient, such as a bed or a wheelchair. Regardless of the manner in which the waste receptacle is positioned and secured in place, the connection between the waste receptacle and the external portion of the catheter is maintained by the flexible discharge conduit which usually takes the form of tubing made of a plastics material.

A problem arises if unintended force, either axial or torsional, is applied to the discharge conduit. Such applications of force can occur, for example, if the patient accidentally catches the conduit on a stationary object while walking, or if the conduit becomes kinked while the patient turns over during sleep. With the balloon fully inflated, the pulling or dislodging of the catheter from its location in the urinary bladder and through the urethra (urinary canal) can cause severe lacerations, bleeding, and the possibility of damage to the external urinary sphincter with permanent urinary incontinence. Any displacement of the balloon also will cause severe pain to the patient. Even if the inflated balloon is not pulled into or through the urinary canal, the mere rotation of the hollow tube within the urinary canal will cause severe pain to the patient.

The problem of pain caused by urinary catheters has been addressed by the prior art. U.S. Pat. No. 4,710,169, the disclosure of which is incorporated herein by reference, discloses an indwelling urinary catheter intended for use by male patients that deals with this problem in several ways. First, the catheter itself includes a collapsible segment that extends substantially the entire length of the urethra. This feature of the invention is stated to provide no longitudinal rigidity so that body movements cannot be translated along the entire length of the catheter. The '169 patent also discloses an external seal that is fitted over the end of the penis. The catheter tube extends through the end of the seal which is defined by a series of accordion folds. The purpose of these folds is to dampen external forces applied to the catheter tube. The '169 patent also addresses the problem of kinking of the discharge tube. Semi-rigid discharge tubes provide “torsional resistance.” The device disclosed in the '169 patent is not usable with female patients, nor is it useful if strong torsional force is applied to the discharge tube.

Another approach is exemplified by U.S. Pat. No. 3,951,153, the disclosure of which is incorporated herein by reference. The '153 patent discloses a safety device in the form of a coupling that connects severed sections of a catheter. Upon the application of sudden force to the catheter, typically by pulling on the conduit connected to the waste receptacle, the coupling separates and thereby prevents the application of further force to the catheter. While the device disclosed in the '153 patent may prevent catastrophic harm to the patient, the separation of the coupling results in the loss of fluid communication with the waste receptacle. Moreover, even if the coupling separates at a relatively low level of force, it is likely that the patient will experience substantial pain. It is doubted that the coupling would prevent the application of torsional forces to the catheter.

It also is known to use external catheters, rather than indwelling catheters. See, for example, U.S. Pat. Nos. 4,378,018, 4,713,066, and 4,846,816, the disclosures of which are incorporated herein by reference. Although such catheters presumably would avoid harm and pain associated with pulling a tube and an attached balloon into or through the urethra, such catheters would not be suitable for use by females. External catheters also have other drawbacks, such as possible leakage, that limit their use even with male patients.

Despite the approaches of the prior art, there remains a need for a device that is usable with a catheter that will prevent or minimize harm and pain to the patient upon the application of axial or torsional forces to the catheter. Any such device hopefully would be inexpensive and would remain usable after axial or torsional forces have been applied thereto. Also, any such device desirably would be usable with catheters of all types, including urinary catheters suitable for use with patients of either sex.

### SUMMARY OF THE INVENTION

In response to the foregoing concerns, the present invention provides a new and improved safety device that is usable with catheters, particularly urinary catheters of the indwelling type. The device according to the invention is usable with a catheter that includes an elongate, flexible tube having a first, internal fluid passageway. The tube has a tip end that in use is inserted into and retained within a body cavity, a central portion that is inserted into and retained within a duct that is in fluid communication with the body cavity, and an exit end that in use is disposed externally of the duct. The tip end includes one or more openings that permit fluid communication between the body cavity and the first fluid passageway. In a typical urinary catheter, an annular inflatable section is spaced inwardly from the tip end, and an input valve is disposed adjacent the discharge end. A second fluid passageway establishes fluid communication between the annular inflatable section and the input valve. The second fluid passageway is separate from the first fluid passageway.

It is expected that the catheter will be connected to a waste receptacle by a discharge conduit. The discharge conduit has a first end connected to the exit end of the catheter and a second end intended to be connected to the waste receptacle. The discharge conduit thus establishes fluid communication between the exit end and the waste receptacle.

The safety device according to the invention comprises a flexible, pleated segment that preferably is included as part of the discharge conduit or the exit end. The pleated segment permits the discharge conduit to be temporarily axially elongated such that the catheter tube will not be moved. Preferably, the pleated segment will be able to stretch at least about 50 percent more than its unstretched length. The pleated segment also permits limited torsional movement of the conduit or the exit end.

The safety device also desirably includes a rotatable, fluid-tight connector that is included as part of the discharge conduit or the exit end. The rotatable connector permits relative rotational movement between the elements to which it is connected, thereby preventing or minimizing the transmission of twisting forces to the catheter tube.

By using the safety device according to the invention, harm and pain caused to the patient that might result from pulling or twisting the discharge conduit can be minimized or eliminated. The device according to the invention will be inexpensive to manufacture and will remain usable after axial or torsional forces have been applied thereto. Also, the device will be usable with catheters of all types, including urinary catheters suitable for use with patients of either sex.

The foregoing and other features and advantages of the invention will be apparent from an examination of the accompanying specification, claims, and drawings.

### BRIEF DESCRIPTION OF THE DRAWINGS

FIG. 1 is a perspective view of a Foley catheter;

FIG. 2 is a cross-sectional view of the catheter of FIG. 1 taken along a plane indicated by line 2-2 in FIG. 1;

FIG. 3 is a side elevation view of the catheter of FIG. 1 showing a balloon portion thereof in an inflated condition;

FIG. 4 is a schematic representation of a Foley catheter connected to an exit conduit and a waste receptacle, and showing a flexible segment and a rotatable connection included as part of the exit conduit;

FIG. 5 is an enlarged view of the flexible segment included as part of the exit conduit; and

FIG. 6 is an enlarged, exploded view of the rotatable connector included as part of the exit conduit.

### DESCRIPTION OF THE PREFERRED EMBODIMENT

Referring generally to the drawings, and particularly to FIG. 4, a safety device according to the invention is indicated by the reference numeral 10. The safety device 10 includes a pleated segment 20 and a rotatable connector 30. The safety device 10 is intended to be used with an indwelling urinary catheter, commonly referred to as a Foley catheter. While the safety device 10 is disclosed in the environment of an indwelling Foley catheter, it can be used in connection with other types of catheters, including external urinary catheters. Indeed, the safety device 10 could be used in any application where it is desired to reduce or eliminate the effects of axial or torsional force that are applied to connected segments of tubing. Accordingly, while the most likely beneficial use of the safety device 10 will be with indwelling urinary catheters, the use of the safety device 10 is not intended to be so limited.

Referring now to FIGS. 1-3, a typical Foley urinary catheter for use with male patients is indicated by the reference numeral 40. Catheters identical or similar to the catheter 40 are commercially available from a number of companies, including Bard Medical Division, 8195 Industrial Boulevard, Covington, Ga. 30014. The catheter 40 includes an elongate, slender tube 42 made of latex, vinyl, silicone, or similar material that defines a first, hollow passageway 44. The tube 42 has a tip end 46, a central portion 48, and an exit end 50. The tube 42 is adapted for insertion into the patient's bladder through his urethra in order to permit urine to be drained from the bladder.

The tip end 46 is provided with a pair of elongated openings 52 that are disposed on opposite sides of the end 46 and which establish fluid communication with the passageway 44. The exit end 50 provides a cylindrical opening for the passageway 44 so that the passageway can be connected to a suitable waste receptacle, as will be described.

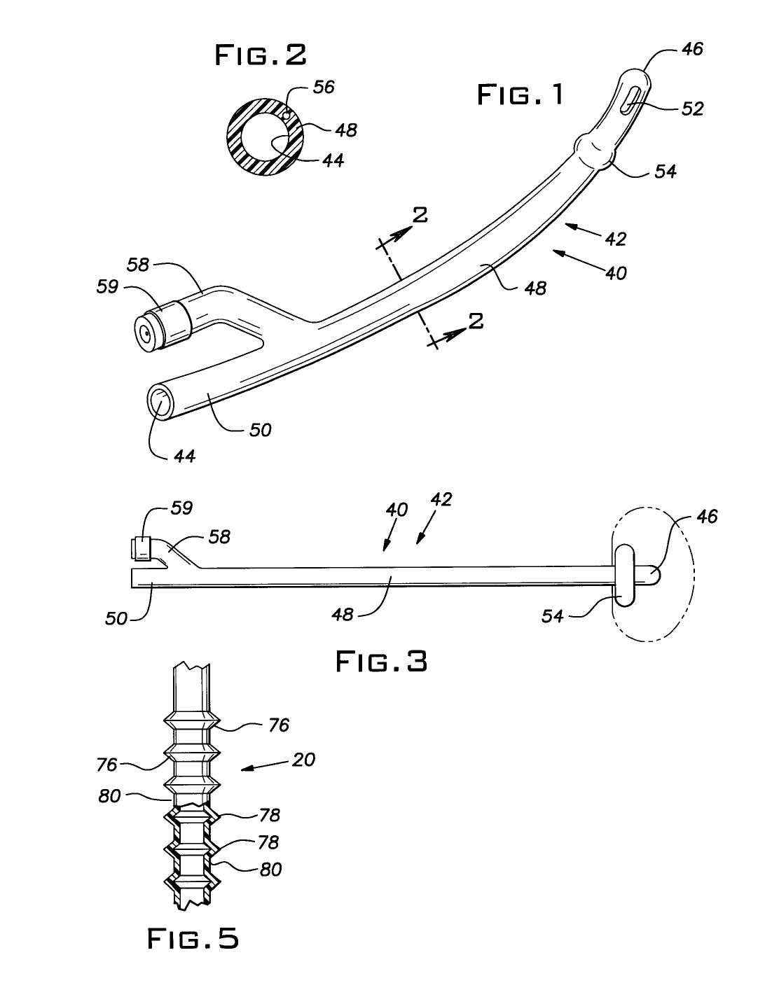

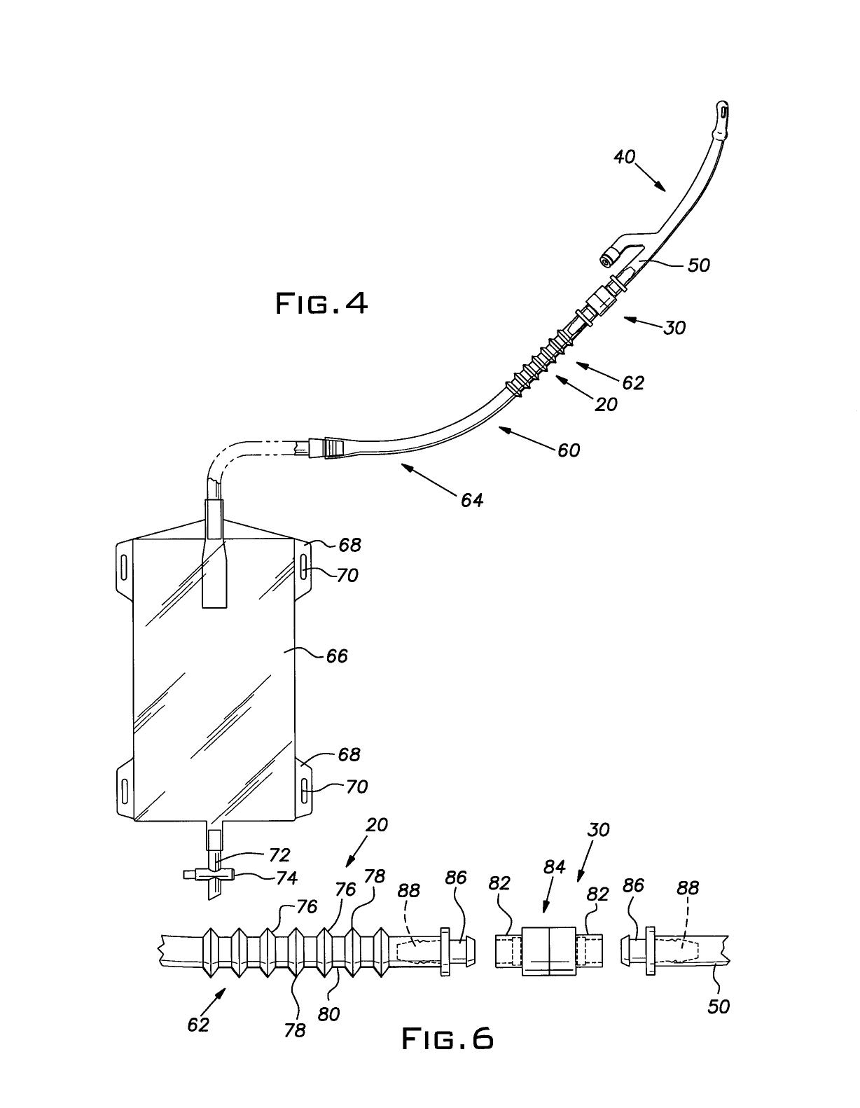

A thin, expansible balloon 54 is disposed adjacent to the openings 52 on the side of the openings 52 closest to the central portion 48. The balloon 54 is connected to the tube 42 by means of a second, hollow passageway 56 (FIG. 2). The second passageway 56 extends through a branch tube 58. A check valve 59 is disposed in the branch tube 58 near the exposed end thereof. The valve 59 is adapted to permit water or air to be introduced into the balloon 54 through the passageway 56. As shown in FIG. 3, the introduction of such water or air into the balloon 54 will cause it to expand into a substantially toroidal shape. The expanded balloon 54 prevents the tip end 46 from being withdrawn unintentionally from the bladder, which is indicated schematically by the dashed lines in FIG. 3.

Referring now to FIG. 4, a discharge conduit 60 includes a first end 62 and a second end 64. The first end 62 is connected to the exit end 50 by means of the rotatable connector 30. The second end 64 is connected to a waste receptacle 66 in the form of a small, flexible bag. The discharge conduit 60 and the waste receptacle 66 are made of a plastics material such as polyurethane or vinyl. The bag 66 includes wings 68 with slots 70, and an outlet 72 having a stopcock 74. The bag can be attached to the patient's leg by the use of suitable straps or tape passed through the slots 70. When necessary, the bag 66 can be drained by opening the stopcock 74. The conduit 60 and the bag 66 are commercially available from Bard Medical Division, 8195 Industrial Boulevard, Covington, Ga. 30014.

The pleated segment 20 that constitutes part of the safety device 10 is included as part of the conduit 60. Preferably, the segment 20 is located in the first end 62, relatively close to the exit end 50. The segment 20 also could be included as part of the exit end 50, if desired. Alternatively, and less desirably, the segment 20 could be located in another portion of the conduit 60 remote from the first end 62. The rotatable connector 30 preferably defines the end of the first end 62, although it, too, could be located elsewhere, such as part of the exit end 50 or in another portion of the conduit 60. If desired, the safety device 10 could include only the pleated segment 20 or only the rotatable connector 30, although the use of both is preferred.

The pleated segment 20 includes a plurality of angled sidewalls 76 that are joined by sharp-edged peaks 78 and valleys 80. The number, thicknesses and length of the sidewalls 76, the peaks 78, and the valleys 80 are chosen to provide a desired percentage of elongation under a sudden, but temporary, axial load. Desirably, the pleated segment will elongate at least approximately 50 percent under load. That is, if the pleated segment in unstressed condition is four inches long, under load it will elongate to at least about six inches. Such a load might be encountered if the patient were to catch the conduit 60 on an unnoticed object while climbing into or out of bed. It is expected that the conduit 60 will remain in a compressed configuration as shown in FIG. 4 under normal operating conditions, i.e., the conduit 60 will support its own weight. However, if the conduit 60 is suddenly pulled or twisted, the segment 20 will elongate and thereby prevent or minimize force from being applied to the exit end 50.

The rotatable connector 30 includes male luer connectors 82 at each end that are joined by a centrally located, rotatable, fluid-tight fitting 84. Female luer connectors 86, each having a barbed end 88, join the male luer connectors 82 to the open ends of the exit end 50 and the conduit 60, respectively. Desirably, the fitting 84 will be sufficiently loose that it will rotate under virtually any torsional load while remaining fluid-tight. The male luer connectors 82 and the fitting 84 are commercially available from Scientific Commodities, Inc., P.O. Box 2458, Lake Havasu City, Ariz. 86406, model no. BB821. Female luer connectors 86 having barbed ends 88 are available from the same company, model no. BB330.

Although the invention has been described in its preferred form with a certain degree of particularity, it will be understood that the present disclosure of the preferred embodiment has been made only by way of example and that various changes may be resorted to without departing from the true spirit and scope of the invention as hereinafter claimed. For example, and not by way of limitation, multiple pleated segments 20 and multiple rotatable connectors 30 could be used. It is intended that the patent shall cover, by suitable expression in the appended claims, whatever features of patentable novelty exist in the invention disclosed.

What is claimed is:

1. In a catheter assembly having an elongate, flexible tube having a first, internal fluid passageway, the tube having a tip end that in use is inserted into and retained within a body cavity, a central portion that is inserted into and retained within a duct that is in fluid communication with the body cavity, and an exit end that in use is disposed externally of the body, the tip end including one or more openings that permit fluid communication between the body cavity and the internal fluid passageway; and
a discharge conduit having a first end and a second end, the first end being connected to the exit end and the second end being remote from the exit end, the discharge conduit permitting fluid to be drained from the body cavity and discharged through the second end, the improvement comprising:
a flexible, pleated segment included as part of a selected one of the discharge conduit or the exit end, the pleated segment in use have a predetermined length, the pleated segment being elongated upon the application of a sudden axial force.

2. The catheter assembly of claim 1, wherein the pleated segment is included as part of the first end of the discharge conduit.

3. The catheter assembly of claim 1, wherein the pleated segment can elongate at least about 50 percent more than the predetermined length.

4. The catheter assembly of claim 1, further comprising:
a rotatable, fluid-tight connector included as part of a selected one of the discharge conduit or the exit end, the fluid-tight connector permitting the discharge conduit to be rotated relative to the exit end.

5. The catheter assembly of claim 4, wherein the rotatable, fluid-tight connector is included as part of the first end of the discharge conduit.

6. The catheter assembly of claim 4, wherein:
the pleated segment is included as part of the first end of the discharge conduit; and
the rotatable connector defines that portion of the first end of the discharge conduit that is connected to the exit end.

7. The catheter assembly of claim 1, further comprising:
an annular inflatable section spaced inwardly from the tip end;
an input valve disposed adjacent the exit end; and
a second fluid passageway that establishes fluid communication between the annular inflatable section and the input valve, the second fluid passageway being separate from the first fluid passageway.

8. The catheter assembly of claim 1, further comprising a waste receptacle, the second end of the discharge conduit being connected to the waste receptacle.

9. A safety device for use with an indwelling catheter having an exit end disposed externally of a patient's body, comprising:
a discharge conduit having a first end and a second end, the first end being connected to the exit end and the second end being remote from the exit end; and
a flexible, pleated segment included as part of a selected one of the discharge conduit or the exit end, the pleated segment in use have a predetermined length, the pleated segment being elongated upon the application of a sudden axial force.

10. The safety device of claim 9, wherein the pleated segment is included as part of the first end of the discharge conduit.

11. The safety device of claim 9, wherein the pleated segment can elongate at least about 50 percent more than the predetermined length.

12. The safety device of claim 9, further comprising:
a rotatable, fluid-tight connector included as part of a selected one of the discharge conduit or the exit end, the fluid-tight connector permitting the discharge conduit to be rotated relative to the exit end.

13. The safety device of claim 12, wherein the rotatable, fluid-tight connector is included as part of the first end of the discharge conduit.

14. The safety device of claim 12, wherein:
the pleated segment is included as part of the first end of the discharge conduit; and
the rotatable connector defines that portion of the first end of the discharge conduit that is connected to the exit end.

15. A urinary catheter assembly, comprising:
an elongate, flexible tube having a first, internal fluid passageway, the tube having a tip end that in use is inserted into and retained within a body cavity, a central portion that is inserted into and retained within a duct that is in fluid communication with the body cavity, and an exit end that in use is disposed externally of the body, the tip end including one or more openings that permit fluid communication between the body cavity and the first fluid passageway;
an annular inflatable section spaced inwardly from the tip end;
an input valve disposed adjacent the discharge end;
a second fluid passageway that establishes fluid communication between the annular inflatable section and the input valve, the second fluid passageway being separate from the first fluid passageway
a waste receptacle;
a discharge conduit having a first end and a second end, the first end being connected to the exit end and the second end being connected to the waste receptacle, the discharge conduit establishing fluid communication between the exit end and the waste receptacle;
a flexible, pleated segment included as part of a selected one of the discharge conduit or the exit end, the pleated segment in use have a predetermined length, the pleated segment being elongated upon the application of a sudden axial force; and
a rotatable, fluid-tight connector included as part of a selected one of the discharge conduit or the exit end, the rotatable, fluid-tight connector permitting that portion of the discharge conduit connected to the exit end to be rotated relative to that portion of the discharge conduit connected to the waste receptacle.

16. The catheter assembly of claim 15, wherein the pleated segment is included as part of the first end of the discharge conduit.

17. The catheter assembly of claim 15, wherein the rotatable, fluid-tight connector is included as part of the first end of the discharge conduit.

18. The catheter assembly of claim 15, wherein:
the pleated segment is included as part of the first end of the discharge conduit; and
the rotatable connector defines that portion of the first end of the discharge conduit that is connected to the exit end.

19. The catheter assembly of claim 15, wherein the pleated segment can elongate at least about 50 percent more than the predetermined length.
